(19) United States
(12) Patent Application Publication
(10) Pub. No.: US 2025/0134901 A1
(43) Pub. Date: May 1, 2025

(54) PHARMACEUTICAL COMPOSITION CONTAINING COMBINATION OF MELOXICAM AND RIZATRIPTAN AND PROCESS OF PREPARATION THEREOF

(71) Applicant: Alembic Pharmaceuticals Limited, Vadodara (IN)

(72) Inventors: Syed Sabir SYED AKHTAR, Medchal District Hyderabad (IN); Barada Prasanna SAHOO, Medchal District Hyderabad (IN); Rahul Sudhakar DABRE, Medchal District Hyderabad (IN)

(73) Assignee: Alembic Pharmaceuticals Limited, Vadodara (IN)

(21) Appl. No.: 18/703,969

(22) PCT Filed: Oct. 20, 2022
(86) PCT No.: PCT/IB2022/060097

§ 371 Date: Apr. 23, 2024

(30) Foreign Application Priority Data
Oct. 26, 2021 (IN) 202121048836

(51) Int. Cl. A61K 31/5415; A61K 9/20; A61K 31/4196

(52) U.S. Cl. CPC A61K 31/5415; A61K 9/2009; A61K 9/2013; A61K 9/2018; A61K 9/2027; A61K 9/2054; A61K 31/4196

(57) ABSTRACT

The present subject matter relates to a solid oral pharmaceutical composition comprising combination of meloxicam and rizatriptan or a pharmaceutically acceptable salt thereof and at least one pharmaceutically acceptable excipient. The present subject matter also relates to a fixed dose pharmaceutical composition comprising meloxicam and rizatriptan or a pharmaceutically acceptable salt thereof and at least one pharmaceutically acceptable excipient. Furthermore, it also relates to methods for manufacturing such compositions and use thereof.

### TECHNICAL FIELD

The present subject matter relates to a solid oral pharmaceutical composition comprising combination of Meloxicam and Rizatriptan or a pharmaceutically acceptable salt thereof and at least one pharmaceutically acceptable excipient. The present subject matter also relates to a fixed dose pharmaceutical composition comprising meloxicam and rizatriptan or a pharmaceutically acceptable salt thereof and at least one pharmaceutically acceptable excipient. Furthermore, it also relates to methods for manufacturing such compositions and use thereof.

### BACKGROUND

Meloxicam, an oxicam derivative, is a member of the enolic acid group of nonsteroidal anti-inflammatory drugs (NSAIDs). It is selective COX-2 inhibitor. It is chemically 4-hydroxy-2-methyl-N-(5-methyl-2-thiazolyl)-2H-1, 2-benzothiazine-3-carboxamide-1, 1-dioxide and is reported to have the following chemical structure:

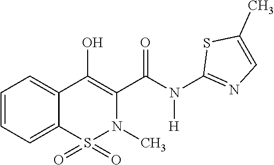 [SMILES: Cc1cnc(NC(=O)C2=C(O)c3ccccc3S(=O)(=O)N2C)s1]

Meloxicam is commercially available in USA as 7.5 mg and 15 mg tablet under the trade name of MOBIC®. It is indicated for relief of the signs and symptoms of osteoarthritis and rheumatoid arthritis, pauciarticular or polyarticular course Juvenile Rheumatoid Arthritis in patients who weigh≥60 kg.

Rizatriptan is a 5-hydroxytryptamine (5-HT1) agonist, second-generation triptan drug. It has the chemical name N,N-dimethyl-2-[5-(1H-1,2,4-triazol-1-ylmethyl)-1H-indol-3-yl]ethanamine, and is reported to have the following chemical structure:

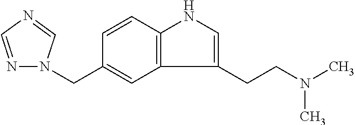 [SMILES: CN(C)CCc1c[nH]c2ccc(Cn3cncn3)cc12]

It was developed by Merck & Co., for the treatment of acute migraine attacks with or without aura. Rizatriptan is available in strengths of 5 and 10 mg as conventional tablets (MAXALT®) and orally disintegrating tablets (MAXALT-MLT®) in the United States.

U.S. Pat. No. 10,517,950 disclose compositions comprising an NSAID such as meloxicam and/or rizatriptan in combination with a cyclodextrin and/or bicarbonate. These compositions may be orally administered to improve the bioavailability or pharmacokinetics of the NSAID for the treatment of pain such as migraine, arthritis, and other conditions.

There is a need in the art for alternative pharmaceutical compositions comprising combination of meloxicam and rizatriptan or a pharmaceutically acceptable salt thereof.

### SUMMARY OF THE SUBJECT MATTER

The present subject matter relates to a solid oral pharmaceutical composition comprising combination of meloxicam and rizatriptan or a pharmaceutically acceptable salt thereof and at least one pharmaceutically acceptable excipient. Furthermore, it also relates to methods for manufacturing such compositions, and uses thereof.

An aspect of the present subject matter is to provide a pharmaceutical composition with improved solubility, dissolution and absorption of meloxicam and rizatriptan or pharmaceutically acceptable salt thereof and a process of manufacturing the pharmaceutical composition thereof.

Meloxicam is considered as BCS Class II drug and has poor aqueous solubility. It has an extended time to reach maximum plasma concentration, or Tmax, which delays its onset of action.

An aspect of the present subject matter is to provide a pharmaceutical composition of meloxicam, rizatriptan or pharmaceutically acceptable salt thereof which has improved solubility of meloxicam and stabilize the meloxicam in acidic pH which further decreases the Tmax of meloxicam i.e less than 3 hours, less than 2 hrs, less than 1 hrs or less than 0.5 hrs. The decrease in Tmax has the benefit for the treatment of pain such as migraine, arthritis and other conditions.

An aspect of the present subject matter is to increase the solubility and bioavailability or decrease the Tmax of meloxicam through the use of a buffering agent and/or a basifying agent and/or a polymer.

Another aspect of the present subject matter is to increase solubility and bioavailability of meloxicam through the use of a buffering agent, a basifying agent (i.e pH modifying agent) and a polymer.

Another aspect of the present subject matter provides a pharmaceutical composition comprising a combination of meloxicam and rizatriptan or a pharmaceutically acceptable salt thereof and at least one pharmaceutically acceptable excipient.

Another aspect of the present subject matter provides a pharmaceutical composition comprising a combination of meloxicam and rizatriptan or a pharmaceutically acceptable salt thereof and a buffering agent.

Another aspect of the present subject matter provides a pharmaceutical composition comprising a combination of meloxicam and rizatriptan or a pharmaceutically acceptable salt thereof and a basifying agent.

Another aspect of the present subject matter provides a pharmaceutical composition comprising a combination of meloxicam and rizatriptan or a pharmaceutically acceptable salt thereof and a polymer.

In one embodiment the present subject matter provides a pharmaceutical composition comprising a combination of meloxicam and rizatriptan or a pharmaceutically acceptable salt thereof and at least one excipient prepared by a solubilization and granulation technique; wherein the solubilized meloxicam is top sprayed on to the substrate excipient by top spray granulation and/or fluidized bed granulation.

Another aspect of the present subject matter provides a pharmaceutical composition comprising a combination of meloxicam and rizatriptan or a pharmaceutically acceptable salt thereof and a buffering agent, a basifying agent, a polymer and/or other excipients prepared by granulation techniques such as top spray granulation and/or fluidized bed granulation.

In one embodiment the present subject matter provides a pharmaceutical composition comprising a combination of meloxicam and rizatriptan or a pharmaceutically acceptable salt thereof and atleast one excipients prepared by granulation technique such as top spray granulation.

Another aspect of the present subject matter provides a process for preparation of pharmaceutical composition by top-spray granulation, comprising the steps of dissolving drug substance and other excipients in a suitable solvent to form granulation solution and spraying the granulation solution onto the fluidized substrate comprising at least one pharmaceutical excipient to form granules. The said granules can be mixed with extra granular excipients and compressed into tablets.

Another aspect of the present subject matter provides a process for preparation of pharmaceutical composition by top-spray granulation, comprising the steps of dissolving drug substance and polymer and/or other excipients in a suitable solvent to form granulation solution and spraying the solution onto the fluidized substrate comprising at least one pharmaceutical excipient, preferably a diluent to form granules. The said granules can be mixed with extra granular excipients and compressed into tablets.

Another aspect of the present subject matter provides a process for preparation of pharmaceutical composition by top-spray granulation, comprising the steps of dissolving drug substance, polymer and basifying agent and/or other excipients in a suitable solvent to form granulation solution and spraying the solution onto the fluidized substrate comprising at least one pharmaceutical excipient preferably diluent to form dispersion granules. The said amorphous dispersion granules can be mixed with extra granular excipients and compressed in to tablets.

Another aspect of the present subject matter provides a pharmaceutical composition comprising a combination of meloxicam and rizatriptan or a pharmaceutically acceptable salt thereof for the treatment of pain including, but not limited to, migraine and other types of headache, inflammatory pain, musculoskeletal pain, neuropathic pain, chronic pain, acute pain, localized pain, systemic pain, cancer-related pain, acute pain, pain due to injury, pain due to illness, post-operative pain, etc.

### DETAILED DESCRIPTION OF THE SUBJECT MATTER

As used herein, “a” or “an” means one or more unless otherwise specified.

The term “Composition” is intended to encompass a product comprising the specified ingredients in the specified amounts, as well as any product which results, directly or indirectly, from combinations of the specified ingredients in the specified amounts.

The term “basifying agent” as used herein refers to a pharmaceutically acceptable excipient which can be utilized to raise the pH of the composition of the invention, leading to enhanced dissolution of the drug.

The term “buffering agent” as used herein refers to a pharmaceutically acceptable excipient which can be utilized to adjust the pH of the composition of the present subject matter leading to enhanced dissolution of the drug. The function of a buffering agent is to drive an acidic or alkaline solution to a certain pH state and prevent a change in the pH.

The term “Intra-granular or Intra-granular portion” as used herein includes, but is not limited to, a part or component of composition mixed or blended to form granules.

The term “Extra-granular or Extra-granular portion” as used herein includes, but is not limited to, a part or component of composition mixed or blended with dry mix formed after granulation, lubricated, and which is further subjected to compression to form the tablets.

Top spray granulation according to the subject matter refers to the process where granules are formed by dissolving the drug substance and/or dissolving polymer and/or other excipients in suitable solvent and/mixture of solvents and spraying the resultant solution onto a fluidized bed substrate. Typically the spraying is performed using an atomizing nozzle. The substrate is maintained in a fluidized state in a fluidized bed while the granulation solution/fluid is sprayed onto the substrate particles. In one aspect, a plurality of substrates is used in the process. The solvent present in the granulation fluid is evaporated resulting in the granules in the fluidized state. The fluidization of the substrate is performed in top-spray apparatus known in the art used to fluidize particles and form a fluidized bed. Generally, the solvent is at least partially or completely evaporated to form the granules. The said granules can be mixed/granulated with extra granular excipients to form the final formulation.

In one embodiment, rizatriptan present in the pharmaceutical composition is in the form of benzoate salt.

In one embodiment, a composition containing meloxicam and rizatriptan or pharmaceutically acceptable salt thereof is prepared by a process comprising following steps,
a) providing a solution of meloxicam, at least one polymer and at least one basifying agent in an inert solvent or mixture of solvents,
b) spraying the resultant solution onto the fluidized bed substrate,
c) simultaneous evaporation or removal of the solvent from the solution obtained in step a), and
d) isolating the granules and mixing them with extragranular material containing rizatriptan to form final blend,
e) compressing the final blend to form tablet.

In one embodiment, the composition of the present subject matter is stable for at least one month under packed condition. In embodiments, the composition of the present subject matter is stable for at least two months under packed condition. In embodiments, the composition of the present subject matter is stable for at least six months under packed condition.

Another aspect of the present subject matter provides a pharmaceutical composition comprising,
(a) meloxicam or a pharmaceutically acceptable salt thereof,
(b) rizatriptan or a pharmaceutically acceptable salt thereof,
(c) one or more pharmaceutically acceptable excipient.

Another aspect of the present subject matter provides a pharmaceutical composition comprising,
(a) meloxicam or a pharmaceutically acceptable salt thereof,
(b) rizatriptan or a pharmaceutically acceptable salt thereof,
(c) one or more pharmaceutically acceptable polymer,
(d) one or more pharmaceutically acceptable basifying agent,
(e) one or more pharmaceutically acceptable buffering agent,
(f) one or more pharmaceutically acceptable diluent,
(g) one or more pharmaceutically acceptable disintegrant,
(h) one or more pharmaceutically acceptable lubricant.

Another aspect of the present subject matter provides a pharmaceutical composition comprising,
(a) meloxicam,
(b) rizatriptan benzoate,
(c) one or more pharmaceutically acceptable polymer,
(d) one or more pharmaceutically acceptable basifying agent,
(e) one or more pharmaceutically acceptable buffering agent,
(f) one or more pharmaceutically acceptable diluent,
(g) one or more pharmaceutically acceptable disintegrant,
(h) one or more pharmaceutically acceptable lubricant.

Another aspect of the present subject matter provides a pharmaceutical composition comprising,
(a) meloxicam or a pharmaceutically acceptable salt thereof,
(b) rizatriptan or a pharmaceutically acceptable salt thereof,
(c) one or more pharmaceutically acceptable excipient,
wherein a pharmaceutical composition is prepared by top spray granulation.

Another aspect of the present subject matter provides a pharmaceutical composition comprising,
(a) meloxicam or a pharmaceutically acceptable salt thereof,
(b) rizatriptan or a pharmaceutically acceptable salt thereof,
(c) one or more pharmaceutically acceptable polymer,
(d) one or more pharmaceutically acceptable basifying agent,
(e) one or more pharmaceutically acceptable buffering agent,
(f) one or more pharmaceutically acceptable diluent,
(g) one or more pharmaceutically acceptable disintegrant,
(h) one or more pharmaceutically acceptable lubricant
wherein a pharmaceutical composition is prepared by top spray granulation.

Another aspect of the present subject matter provides a pharmaceutical composition comprising,
(a) meloxicam,
(b) rizatriptan benzoate,
(c) one or more pharmaceutically acceptable polymer,
(d) one or more pharmaceutically acceptable basifying agent,
(e) one or more pharmaceutically acceptable buffering agent,
(f) one or more pharmaceutically acceptable diluent,
(g) one or more pharmaceutically acceptable disintegrant,
(h) one or more pharmaceutically acceptable lubricant,
wherein a pharmaceutical composition is prepared by top spray granulation.

According to one embodiment, the excipients include one or more of polymers, basifying agents, buffering agents, disintegrants, binders, surfactants, fillers, and lubricants.

In this specification the terms “polymer” and “polymers” are intended to be interpreted in the context of pharmaceutical formulation science. Suitable polymer according to the present subject matter can be selected form the group of, but not limited to polyvinyl pyrrolidone (PVP), Soluplus, Povidone K-30, Povidone K-90, polyvinylpyrrolidone vinyl acetate, co-povidone, polyvinylacetal diethylaminoacetate (AEA®), polyvinyl acetate phthalate, polyoxyethylene-polyoxypropylene copolymers (Poloxamer® 188), polyoxyethylene (40) stearate, polyethylene glycol monomethyl ether, polyethylene glycol, poloxamer 188, pluronic F-68, methylcellulose, methacrylic acid copolymer (Eudragit or Eudragit-RLPO), hydroxypropylmethyl cellulose (HPMC), hydroxypropyl cellulose (HPC), hydroxypropylmethyl cellulose phthalate, hydroxypropylmethyl cellulose acetate succinate (HPMC-AS), hydroxypropylmethyl cellulose, hydroxypropyl cellulose SSL (HPC-SSL), hydroxypropyl cellulose SL (HPC-SL), hydroxypropyl cellulose L (HPC-L), hydroxyethyl cellulose, Soluplus® (polyvinyl caprolactam-polyvinyl acetate-polyethylene glycol graft copolymer (PCL-PVAc-PEG)), gelucire 44/14, ethyl cellulose, D-alpha-tocopheryl polyethylene glycol 1000 succinate, cellulose acetate phthalate, carboxymethyl ethylcellulose and the like.

The term “buffering agent” is intended to be interpreted in the context of pharmaceutical formulation science. Suitable buffering agent/s can be selected from the group of, but not limited to a carbonate, derivatives, or salts thereof. Examples of carbonates may include aluminum carbonate, ammonium carbonate, barium carbonate, calcium carbonate, cobalt carbonate, lanthanum carbonate, lithium carbonate, magnesium carbonate, magnesium oxide, manganese carbonate, potassium carbonate, sodium citrate, sodium carbonate or combinations thereof.

The term “basifying agent” is intended to be interpreted in the context of pharmaceutical formulation science. Suitable basifying agent/s can be selected from the group of, but not limited to a meglumine, sodium hydroxide, calcium hydroxide, potassium hydroxide or combinations thereof.

The term “diluent” is intended to be interpreted in the context of pharmaceutical formulation science. Suitable diluents can be selected from the group of, but not limited to microcrystalline cellulose (MCC), pregelatinized starch, sugar and sugar alcohol such as mannitol, lactose, fructose, isomalt, lactitol, maltitol, sorbitol, erythritol, maltose, polydextrose, sucrose, trehalose and xylitol; calcium carbonate, dicalcium phosphate, calcium sulfate, cellulose acetate, ethylcellulose, inulin, magnesium carbonate, magnesium oxide, maltodextrin, sodium bicarbonate, sodium carbonate and sodium chloride.

The term “binder” is intended to be interpreted in the context of pharmaceutical formulation science. Suitable binder can be selected from the group of, but not limited to povidone, copovidone pregelatinized starch, cellulose, methyl cellulose, ethyl cellulose, cellulose derivatives (e.g., hydroxypropyl methyl cellulose (HPMC), hydroxypropyl cellulose (HPC)), polyethylene glycol and hydroxypropyl methyl cellulose acetate succinate (HPMC-AS).

The terms “disintegrant” and “disintegrants” are intended to be interpreted in the context of pharmaceutical formulation science. Suitable disintegrants according to the present subject matter can be selected from but not limited to Crospovidone, croscarmellose sodium (CCS), sodium starch glycolate (SSG), pregelatinized starch, alginic acid, sodium alginate, carboxymethylcellulose calcium, chitosan, crospovidone, glycine, guar gum, hydroxypropyl cellulose, low-substituted hydroxypropyl cellulose, magnesium aluminum silicate, methylcellulose, povidone, sodium alginate, sodium carboxymethylcellulose and starch.

The terms “surfactant” and “surfactants” are intended to be interpreted in the context of pharmaceutical formulation science. Suitable pharmaceutical surfactant according to the present subject matter can be selected from but not limited to sodium lauryl sulfate (SLS), poloxamers, polysorbate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine and the like.

The terms “lubricant” and “lubricants” are intended to be interpreted in the context of pharmaceutical formulation science. Suitable pharmaceutical lubricant according to the present subject matter can be selected from but not limited to magnesium, aluminium, zinc or calcium stearate, glyceryl behenate, glyceryl dibehenate, glyceryl monostearate, glyceryl palmitostearate, a mixture of behenate esters of glycerine (e.g. a mixture of glyceryl behenate, tribehenin and glyceryl behenate), magnesium stearate, myristic acid, palmitic acid, stearic acid, talc, tribehenin, sodium stearyl fumarate (SSF) and sucrose stearate.

Still another embodiment of the present subject matter provides a composition wherein the organic solvent used is selected from isopropyl alcohol, ethanol, methanol, acetone, dichloromethane, acetonitrile, petroleum ether or mixture thereof.

As used herein, the word “comprising” describes components that must be present, but leaves open the possibility that other unspecified components may also be present within the scope of the relevant term.

In one aspect there is provided a pharmaceutical tablet comprising the pharmaceutical composition as defined herein.

In one embodiment there is provided a pharmaceutical tablet comprising a pharmaceutical composition in the form of tablet core, wherein the tablet core is coated with a layer of coating. In one embodiment the coating is a film coating.

When the tablet has a film coating, the film coating may be applied using conventional methods. A coating can be used to provide protection against, for example, moisture ingress or degradation by light, to colour the formulation, or to modify or control the release of meloxicam and rizatriptan from the formulation.

In one embodiment, the pharmaceutical composition is a pharmaceutical tablet composition (for oral administration).

In one embodiment, the pharmaceutical composition is a pharmaceutical tablet composition suitable for oral administration to a human.

In one embodiment, the pharmaceutical composition is a pharmaceutical tablet composition suitable for oral administration for the treatment of pain including, but not limited to, migraine and other types of headache, inflammatory pain, musculoskeletal pain, neuropathic pain, chronic pain, acute pain, localized pain, systemic pain, cancer-related pain, acute pain, pain due to injury, pain due to illness, post-operative pain, etc.

In one embodiment, the pharmaceutical tablet comprises 5 mg, 7.5 mg, 10 mg, 15 mg and 20 mg meloxicam or its salt thereof.

In one embodiment, the pharmaceutical tablet comprises 5 mg and 10 mg rizatriptan free base which is equivalent to 7.265 mg and 14.53 mg of rizatriptan benzoate respectively.

In one embodiment, the pharmaceutical tablet comprises a fixed dose combination of 15 mg meloxicam or its salt thereof and 10 mg rizatriptan or its salt thereof.

In one embodiment, the pharmaceutical tablet comprises a fixed dose combination of 20 mg meloxicam or its salt thereof and 10 mg rizatriptan or its salt thereof.

In a further aspect, there is provided the use of a pharmaceutical composition, as defined herein, for the manufacture of a medicament.

In one embodiment, there is provided the use of a pharmaceutical composition, as defined herein, for the manufacture of a medicament for the treatment of pain including, but not limited to, migraine and other types of headache, inflammatory pain, musculoskeletal pain, neuropathic pain, chronic pain, acute pain, localized pain, systemic pain, cancer-related pain, acute pain, pain due to injury, pain due to illness, post-operative pain, etc.

In one aspect, there is provided a pharmaceutical composition, as defined herein, for use as a medicament.

In one embodiment, a pharmaceutical composition is a pharmaceutical tablet.

In one embodiment, there is provided a method of treatment of migraine.

In one embodiment, the composition containing combination of meloxicam and rizatriptan can be in the form of solid dispersion prepared by employing techniques known in the art or procedures described or exemplified in any aspect of the instant application.

It is to be understood that each of the variously stated ranges is intended to be continuous so as to include each numerical parameter between the stated minimum and maximum value of each range.

Embodiments provided herein may be more fully understood by reference to the following examples. These examples are meant to be illustrative of pharmaceutical composition and dosage form provided herein, but is not in any way limiting.

### Example 1

TABLE 1
Pharmaceutical composition of meloxicam and rizatriptan

| | | % of ingredient by |
|---|---|---|
| Sr. No. | Ingredients | weight of core tablet |
| 1 | Meloxicam or salt thereof | 2-6 |
| 2 | Rizatriptan or salt thereof | 1-4 |
| 3 | Polymer (e.g. Povidone, Copovidone, | 0.25-5 |
| | HPMC-AS, HPMC, HPC) | |
| 4 | Buffering agent (e.g. Calcium carbonate | 10-60 |
| | Magnesium carbonate, Magnesium oxide) | |
| 5 | Basifying agent (e.g. Meglumine, sodium | 1-10 |
| | hydroxide) | |
| 6 | Diluents (e.g. Mannitol, microcrystalline | 10-70 |
| | cellulose, lactose) | |
| 7 | Disintegrant (e.g. Sodium starch glycolate, | 1-10 |
| | crospovidone, croscarmellose sodium, | |
| | L-substituted hydroxypropyl cellulose) | |
| 8 | Binder (e.g. Povidone, copovidone) | 0.25-5 |
| 9 | Surfactant (e.g. Polysorbate, sodium lauryl | 0-5 |
| | sulphate) | |
| 10 | Lubricant(e.g. Magnesium stearate, stearic | 0.25-5 |
| | acid, sodium stearyl fumarate) | |
| 11 | Optionally film coating | 2-5 |

### Example 2

Table 2 provided the experimental solubility data in different pH buffered solutions.

TABLE 2
Solubility data of meloxicam

| | | Meloxicam API Solubility |
|---|---|---|
| Sr. No. | Media | (BCS) (mg/ml) |
| 1 | 0.01N HCl (pH 2.0) | 0.02 |
| 2 | pH 4.5 Acetate buffer | 0.03 |
| 3 | pH 6.8 Phosphate Buffer | 2.70 |
| 4 | pH 7.5 Phosphate buffer | 0.11 |

### Example 3

Solubility study of meloxicam was conducted using basifying agents (like meglumine, sodium hydroxide) in aqueous, hydro-alcoholic and/or non-aqueous vehicles. A clear solution of meloxicam is desired to solubilize the drug. Meloxicam with meglumine provided a clear solution in aqueous system with homogenization. Meloxicam with meglumine and sodium hydroxide provided a clear solution in hydro-alcoholic system (water and methanol).

TABLE 3
Solubility study and pH measurement using meglumine and sodium
hydroxide

| Sr. | | | Observation of |
|---|---|---|---|
| No. | Ingredients | pH | meloxicam solution |
| 1 | Meloxicam (20 mg) + meglumine | 13.72 | Dispersion |
| | (25 mg) + NaOH (7 mg) + water | | |
| 2 | Meloxicam (20 mg) + meglumine | 10.24 | Dispersion |
| | (25 mg) + water | | |
| 3 | Meloxicam (20 mg) + meglumine | 10.15 | Clear yellow |
| | (25 mg) + water (with homogenization) | | colored solution |
| 4 | Meloxicam (20 mg) + meglumine | 13.78 | Clear yellow |
| | (25 mg) + NaOH (7 mg) + water + | | colored solution |
| | methanol | | |

### Example 4

TABLE 4
Granulation

| S. | | |
|---|---|---|
| No | Name of the Ingredients | Qty. % w/w |
| | Intra granular Part | |
| 1 | Mannitol | 66.68 |
| 2 | Crospovidone | 5.00 |
| | Meloxicam solution | |
| 3 | Meloxicam | 2.50 |
| 4 | Meglumine | 4.16 |
| 5 | Methanol | Q.S |
| 6 | Purified Water | Q.S |
| | Extra granular | |
| 7 | Mannitol | 20.00 |
| 8 | Sodium Stearyl fumarate | 1.66 |
| | Total | 100.00 |

### Procedure for Example 4

1. Meloxicam solution was prepared by dissolving meloxicam and meglumine in water and methanol.
2. Mannitol and crospovidone were mixed together and granulated using meloxicam solution of step 1 by granulation.
3. The granules obtained in step 2 were mixed with mannitol and sodium stearyl fumarate and compressed into tablet.

### Example 5

TABLE 5
Top Spray Granulation

| S. | | |
|---|---|---|
| No. | Name of the Ingredients | Qty. % w/w |
| | Intra granular Part | |
| 1 | Mannitol | 37.37 |
| 2 | Microcrystalline cellulose | 36.16 |
| | Meloxicam solution | |
| 3 | Meloxicam | 2.5 |
| 4 | Meglumine | 4.16 |
| 5 | Sodium hydroxide | 1.16 |
| 6 | Povidone K30 | 1.66 |
| 7 | Methanol | Q.S |
| 8 | Purified Water | Q.S |
| | Extra granular | |
| 9 | Rizatriptan benzoate | 2.42 |
| 10 | Microcrystalline cellulose | 10.07 |
| 11 | Crospovidone | 2.5 |
| 12 | Magnesium stearate | 2.00 |
| | Total | 100.00 |

### Procedure for Example 5

1. Meloxicam solution was prepared by dissolving meloxicam, meglumine, sodium hydroxide and povidone K30 in water and methanol.
2. Mannitol and microcrystalline cellulose were mixed together and granulated using meloxicam solution of step 1 by top spray granulation.
3. The granules obtained in step 2 were mixed with rizatriptan benzoate, microcrystalline cellulose, crospovidone, magnesium stearate and compressed into tablet.

### Example 6, 7, 8 and 9

TABLE 6
Top Spray Granulation

| | | Example | Example | Example | Example |
|---|---|---|---|---|---|
| | | 6 | 7 | 8 | 9 |
| S. | Name of the | Qty. | Qty. | Qty. | Qty. |
| No: | Ingredients | % w/w | % w/w | % w/w | % w/w |
| | Intra granular | | | | |
| | Part | | | | |
| 1 | Mannitol | 37.35 | 41.69 | 43.33 | 43.33 |
| 2 | Microcrystalline | 36.16 | — | — | — |
| | cellulose | | | | |
| 3 | Calcium carbonate | — | 33.33 | 33.33 | 33.33 |
| 4 | Crospovidone | — | — | 5.00 | 5.00 |
| | Meloxicam | | | | |
| | solution | | | | |
| 5 | Meloxicam | 2.50 | 2.50 | 2.50 | 2.50 |
| 6 | Meglumine | 4.17 | 4.17 | 4.17 | 4.17 |
| 7 | Sodium hydroxide | 1.16 | 1.16 | — | — |
| 8 | Povidone K90 | 1.66 | 1.66 | 0.83 | — |
| 9 | Povidone K30 | — | — | — | 1.66 |
| 10 | Methanol | Q.S | Q.S | Q.S | Q.S |
| 11 | Purified water | Q.S | Q.S | Q.S | Q.S |
| | Extra granular | | | | |
| 12 | Microcrystalline | 12.50 | 10.00 | — | — |
| | cellulose | | | | |
| 13 | Mannitol | | | 6.68 | 4.68 |
| 14 | Crospovidone | 2.50 | 3.33 | 2.50 | 3.33 |
| 15 | Magnesium | 2.00 | 2.16 | 1.66 | 2.00 |
| | stearate | | | | |

| | | | | |
|---|---|---|---|---|
| Total | 100.00 | 100.00 | 100.00 | 100.00 |

### Procedure for Example 6 to 9 is Similar to Example 5

### Example 10, 11 and 12

TABLE 7
Top Spray Granulation

| S. | Name of the | Example 10 | Example 11 | Example 12 |
|---|---|---|---|---|
| No. | Ingredients | Qty. % w/w | Qty. % w/w | Qty. % w/w |
| | Intra granular Part | | | |
| 1 | Mannitol | 38.11 | 43.33 | 40.11 |
| 2 | Calcium carbonate | 33.33 | 33.33 | 33.33 |
| 3 | Crospovidone | 5.00 | 5.00 | 5.00 |
| | Meloxicam solution | | | |
| 4 | Meloxicam | 3.33 | 2.50 | 3.33 |
| 5 | Meglumine | 4.16 | 4.17 | 4.16 |
| 6 | PVP K30 | 1.66 | — | — |
| 7 | PVP K90 | — | 0.83 | 0.83 |
| 8 | Methanol | Q.S | — | — |
| 9 | Purified water | Q.S | Q.S | Q.S |
| | Extra granular | | | |
| 10 | Rizatriptan benzoate | 2.42 | — | 2.42 |
| 11 | Mannitol | 6.66 | 6.68 | 6.66 |
| 12 | Crospovidone | 3.33 | 2.50 | 2.50 |
| 13 | Magnesium stearate | 2.00 | 1.66 | 1.66 |

| | | | |
|---|---|---|---|
| Total | 100.00 | 100.00 | 100.00 |

### Procedure for Example 10, 11 and 12

1. Meloxicam solution was prepared by dissolving meloxicam, meglumine, and povidone K30/K90 in water and/or methanol.
2. Mannitol, calcium carbonate and crospovidone were mixed together and granulated using meloxicam solution of step 1 by top spray granulation.
3. The granules obtained in step 2 were mixed with rizatriptan benzoate, mannitol, crospovidone, magnesium stearate and compressed into tablet.

### Example 13—Dissolution Study

The dissolution described herein was performed according to the general procedure of the United States Pharmacopoeia using Apparatus II (Paddle), at 75 rpm with 500 ml of 0.01N HCl media at a temperature of 37° C. The samples were withdrawn at 15 minutes and drug release was determined. The results are presented in Table 8.

TABLE 8

| | Dissolution Data in pH 2.0, 500 ml, Paddle, 75 rpm | |
|---|---|---|
| | (0.01N HCl) (Drug Release in 15 min) | |
| Example | Meloxicam | Rizatriptan |
| 4 | 35% | — |
| 5 | 75% | — |
| 6 | 67% | — |
| 7 | 87% | — |
| 8 | 88% | — |
| 9 | 89% | — |
| 10 | 91% | 94% |
| 11 | 89% | — |
| 12 | 93% | 98% |

### Example 14

TABLE 9
Top Spray Granulation

| S. | | |
|---|---|---|
| No. | Name of the Ingredients | Qty. % w/w |
| | Intra granular Part | |
| 1 | Mannitol | 33.33 |
| 2 | Calcium carbonate | 44.00 |
| 3 | Crospovidone | 2.50 |
| | Meloxicam solution | |
| 4 | Meloxicam | 3.33 |
| 5 | Meglumine | 4.17 |
| 6 | PVP K30 | — |
| 7 | PVP K90 | 0.83 |
| 8 | Methanol | — |
| 9 | Purified water | Q.S |
| | Extra granular | |
| 10 | Rizatriptan benzoate | 2.42 |
| 11 | Mannitol | 6.66 |
| 12 | Crospovidone | 1.67 |
| 13 | Magnesium stearate | 1.09 |
| | Total | 100.00 |

### Procedure for Example 14

1. Meloxicam solution was prepared by dissolving meloxicam, meglumine, and povidone K90 in water.
2. Mannitol, calcium carbonate and crospovidone were mixed together and granulated using meloxicam solution of step 1 by top spray granulation.
3. The granules obtained in step 2 were mixed with rizatriptan benzoate, mannitol, crospovidone, magnesium stearate and compressed into tablet.

### Example 15—Dissolution Study

The dissolution described herein was performed using USP Apparatus II (Paddle), at 75 rpm with 900 ml of phosphate buffer pH 7.5 media at a temperature of 37° C. for meloxicam and USP Apparatus II (Paddle), at 50 rpm with 500 ml of 0.1N HCl at a temperature of 37° C. for Rizatriptan. The samples were withdrawn at 15 minutes and drug release was determined. The results are presented in Table 10.

TABLE 10

| | Dissolution Data | |
|---|---|---|
| | Meloxicam | |
| | pH 7.5 Phosphate Buffer, | Rizatriptan |
| | 900 mL, USP Apparatus II, | 0.1N HCl, 500 mL, USP |
| Example | 75 RPM | Apparatus II, 50 RPM |
| 14 | 94% | 99% |

### Example 16: In-Vivo Pharmacokinetic Study

The tablet of Example 9 (T1) and 11 (T2) was administered to 22 human subjects. Plasma samples were collected for concentration analysis of meloxicam at several time points. Concentrations of meloxicam were determined using suitable technique. Pharmacokinetic parameters were calculated. The results for meloxicam were similar to that of example 9 and 11. The geometric mean Tmax of meloxicam in Example 9 and Example 11 was found to be 0.5 hours in fasted condition as compare to 4.5 hrs for Mobic (commercial product) as per literature data.

TABLE 11
Pharmacokinetic data (Fasting Data for T1 and T2 formulation)

| | | | | T (1/2) | | Cmax | | T max* | |
|---|---|---|---|---|---|---|---|---|---|
| | Statistic | AUC inf. | | Hr. | | (ng/ml). | | (Hr.) | |
| Meloxicam | N | 22 | | 22 | | 22 | | 22 | 22 |
| Tab | Test | T1 | T2 | T1 | T2 | T1 | T2 | T1 | T2 |
| 15 mg | Geometric | 62346 | 64529 | 22 | 21 | 2477 | 2634 | 0.5 | 0.5 |
| | mean | | | | | | | | |
| | SD | 36746 | 34853 | 12 | 10 | 505 | 538 | 0.3-3.0 | 0.3-4.5 |

*Tmax presented as median and range

1. A pharmaceutical composition comprising a combination of meloxicam and rizatriptan or a pharmaceutically acceptable salt thereof and at least one pharmaceutically acceptable excipient.

2. The pharmaceutical composition as per claim 1, wherein pharmaceutically acceptable excipient is selected from the group consisting of a polymer, a buffering agent, a basifying agent, a diluent, a binder and a disintegrant.

3. The pharmaceutical composition as per claim 1, wherein the composition is prepared by top spray granulation.

4. The pharmaceutical composition as per claim 1, wherein the composition comprises a fixed dose combination of a) meloxicam and rizatriptan benzoate b) one or more polymer c) one or more basifying agent, d) one or more buffering agent and e) one or more disintegrant.

5. A process for preparation of composition comprising combination of meloxicam and rizatriptan benzoate as per claim 3 comprises:
a) providing a solution of meloxicam, at least one polymer and at least one basifying agent in an inert solvent or mixture of solvents,
b) spraying the resultant solution onto the fluidized bed substrate,
c) simultaneous evaporation or removal of the solvent from the solution obtained in step a), and
d) isolating the granules and mixing them with extragranular material containing rizatriptan benzoate to form final blend,
e) compressing the final blend to form tablet.
